(19) United States
(12) Patent Application Publication
(10) Pub. No.: US 2006/0241693 A1
(43) Pub. Date: Oct. 26, 2006

(54) Plastic mono-filamentary thermo-contractible surgical thread

(76) Inventors: Carlos Alberto Peregrina, Buenos Aires (AR); Marcelo Gustavo Visentini, Buenos Aires (AR); Roberto Felisati, Buenos Aires (AR)

Correspondence Address: EPSTEIN DRANGEL BAZERMAN & JAMES, LLP, 60 EAST 42ND STREET, SUITE 820, NEW YORK, NY, 10165, US

(21) Appl. No.: 11/176,845

(22) Filed: Jul. 6, 2005

(30) Foreign Application Priority Data
Apr. 26, 2005 (AR) 20050101643

(51) Int. Cl. A61L 17/00

(52) U.S. Cl. 606/228

(57) ABSTRACT

When introduced into the body, the plastic mono-filamentary thermo-contractible surgical thread retracts in response to the application of heat and when interacting with adjacent tissues, causes them to retract, tightening same.

### TECHNICAL FIELD OF THE INVENTION

The present invention is to be utilized in human organisms and vertebrate animals, where tissue retraction or contraction would be useful, as it is the retraction of stretched and aged skin, of the mucous membranes, the contraction and closing of injuries, the light diminution of the tubular organs, etc.

### BACKGROUND OF THE INVENTION

The passage of time produces the loss of skin elasticity through a gradual dehydration process, the elastic fiber denaturalizing, collagen decrease, etc. This process is enhanced by sunlight action, environmental pollution, stress and nicotine addiction among other factors.

Skin is all over body deep tissues. Skin elasticity loss leads to skin stretching or lengthening. This results in a larger skin surface to cover the same initial deep tissue area. Skin therefore folds or wrinkles. Gravity makes skin folds fall vertically in erect position. The final effect is face and other body parts aging aspect.

There have been a good many procedures attempting to stop or diminish this aging process, aiming thus to achieve an all-time young look. On the one hand, aesthetic surgery targets to highly attenuate aging consequences in different parts of the body such as face, abdomen, bust by lifting, stretching and resectioning skin surplus; i.e. stretched skin is not shortened. On the contrary, it is stretched even more and what remains is removed in order to cover the deep tissues and hence achieve an outer skin smoother aspect. Surgery succeeds in skin smoothing. However, surgery fails to improve skin structure. Furthermore, ever lasting scars must be considered.

Dermatological procedures, on the other hand, such as peelings, massotherapy, nourishing creams, collagen applications, attempt to restore skin original structure, stimulating it so that it can recover its thickness and former features. They succeed in maintaining skin health. However, they fail to lift major folds due to the passage of time; i.e. they fail to retract stretched skin.

At present there are different threads which tend to pull skin towards a desired direction, used basically for aesthetic purposes to endeavor to lift or thwart the effect of gravity over human skin and hence disguise or attenuate folds and wrinkles. They are threads with small spicules (hook-liked) which are one-way-only placed under the skin. When pulled by the extreme in the opposed direction they should lift skin folds, translating skin surplus to higher less noticeable areas. Ordinary surgical threads fixed by means of knots, at one end, to the skin deep layer or to other deep support tissues, at the other end, are often utilized as well, so as to enable the lifting or traction of some specific skin points such as the extreme of the eyebrow or the corners of the mouth. Other threads are left under the skin for the body to fix them through a reaction in self defense called ‘reaction to a strange organism’. This reaction attempts to restrain skin stretching process. Taking into account the above mentioned techniques, the present invention does not pursue skin traction (and consequently its stretching), but skin and/or mucous membranes retraction. In other words, it pursues to reduce the dimension of skin and/or mucous membrane exposed surface. Or in a longitudinal sense, to achieve that time-going-by stretching returns to its original dimension. Our invention reassures skin surplus retraction at will through subcutaneous or submucous implant of different amount of thermo-contractible surgical threads, which when being externally stimulated by different amount of caloric energy regulated at the moment and in time, produce their contraction. This contraction is transmitted to supradjacent skin or mucous membranes causing its retraction at will.

This same principle, i.e. the property of heat stimulated thread contraction, can be applied to other areas of the body to achieve beneficial effects such as internal light (space) reduction of tubular organs, suture knot tension increase to improve injury border closing, blood vessel or Falopian tube ligature gauging, etc.

### BRIEF DESCRIPTION OF THE INVENTION

The aim of the present invention is to dispose of a plastic mono-filamentary thermo-contractible surgical thread which, when introduced in the body, would be retractable to heat application and when interacting with surrounding tissues, would transfer this property to them, dragging up with it the involved body parts.

### BRIEF DESCRIPTION OF THE DRAWINGS

To serve as an example in order to contribute to the understanding of the invention aim, five figures are being detailed below:

FIG. 1 is a simple view of the thread, with its external configuration, not differing from any other surgical thread.

FIG. 2 is an outline of the under skin or mucous membrane implanted thread at the moment of its placement.

FIG. 3 exemplifies how strange corpse reaction is produced totally surrounding the thread and attaching it to adjacent tissues.

FIG. 4 sketches the shortening reaction of the thread and its surroundings when being heat stimulated.

FIG. 5 is a outline which shows the relationship between skin surface to be covered and the dimension of the skin that covers it (stretched and wrinkly, and retracted and smooth)

### DETAILED DESCRIPTION OF THE INVENTION

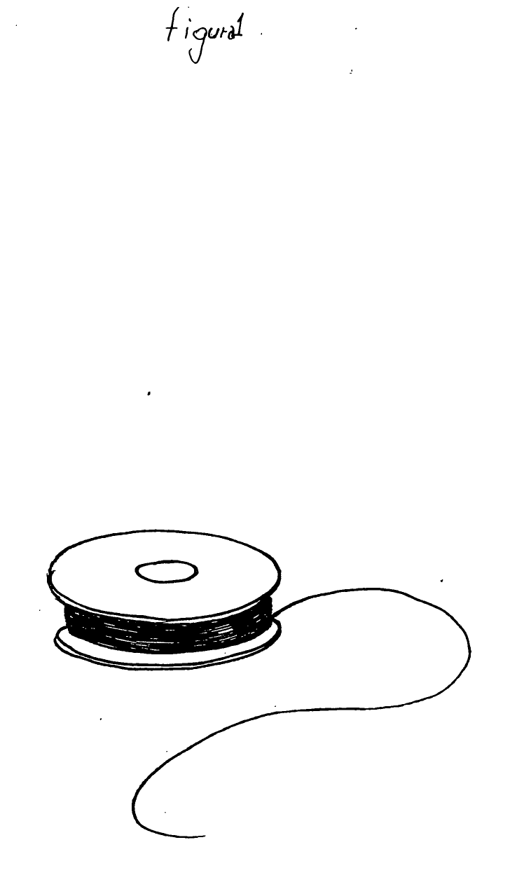

The present invention relates to a plastic mono-filamentary thermo-contractible surgical thread as shown in FIG. 1.

To produce the surgical thread the following materials, raw materials, tools, process, etc are necessary.

Extrusion Equipment:

Extrusor:

Screw diameter: 30 mm

Relation: L/D 22

Geometry: universal for polyamides.

Heating: 4 areas trough resistance

Row:

Diameter: 1.00 mm

Stretchers:

With hot air tunnels

Material used:

Polyamide 6 relative viscosity (dl. SO4H2) between 5 and 5.2

Atoxic colorant of allowed use.

Extrusor:

Zone 1: 245° C.

Zone 2: 275° C.

Zone 3: 270° C.

Zone 4: 270° C.

RPM: 11.00

Cooling basin:

H2O to 30° C./35° C.

Exit speed:

23.60 m/min.

Orientation:

1^{st} stage: relation 4.10:1 in tunnel to 130° C.

2^{nd} stage: relation 1.03:1 in tunnel to 160° C.

Under these process conditions an oriented colored monofilament, smooth and able to retract to the later heat application. The thread is gathered in small plastic reels. (1 kg. portage)

Sterilization

After the thread industrial process has been carried out, the thread should undergo a sterilization which will allow the innocuous use of the present invention. Any sterilization system not exceeding 50° C. can be suitable.

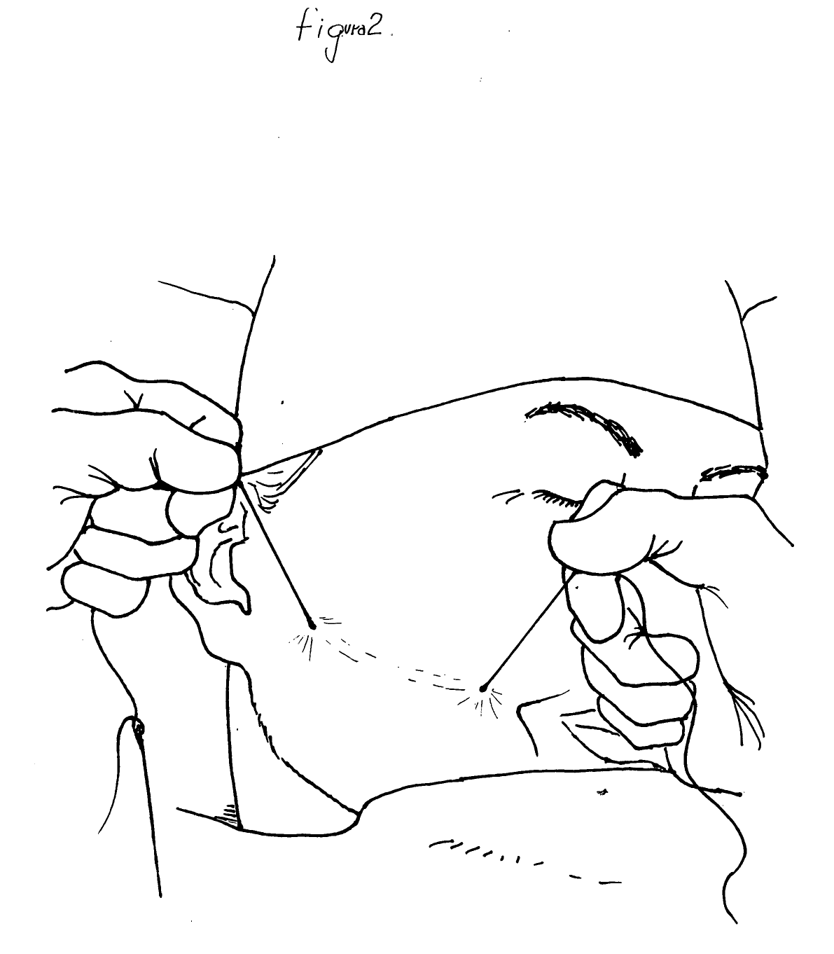

Afterwards the thread is threaded into a straight needle preferably hollow and introduce by skin or mucous membrane puncture under local anesthesia and sterilized conditions. The needle slides subdermically or submucously parallel to the skin surface all over the needle extension, exiting out as shown in FIG. 2. Once the needle has been extracted, the thread slides over the created space and is cut off at the level of skin entrance and exit. Skin starts moving in both places (entrance and exit) till thread ends disappear under the skin, so that the thread is left under the skin (subdermis thread or submucous thread). In this way, the thread can be introduced in multiple directions an in variable quantities, making up a network or weave. No special cares are required afterwards.

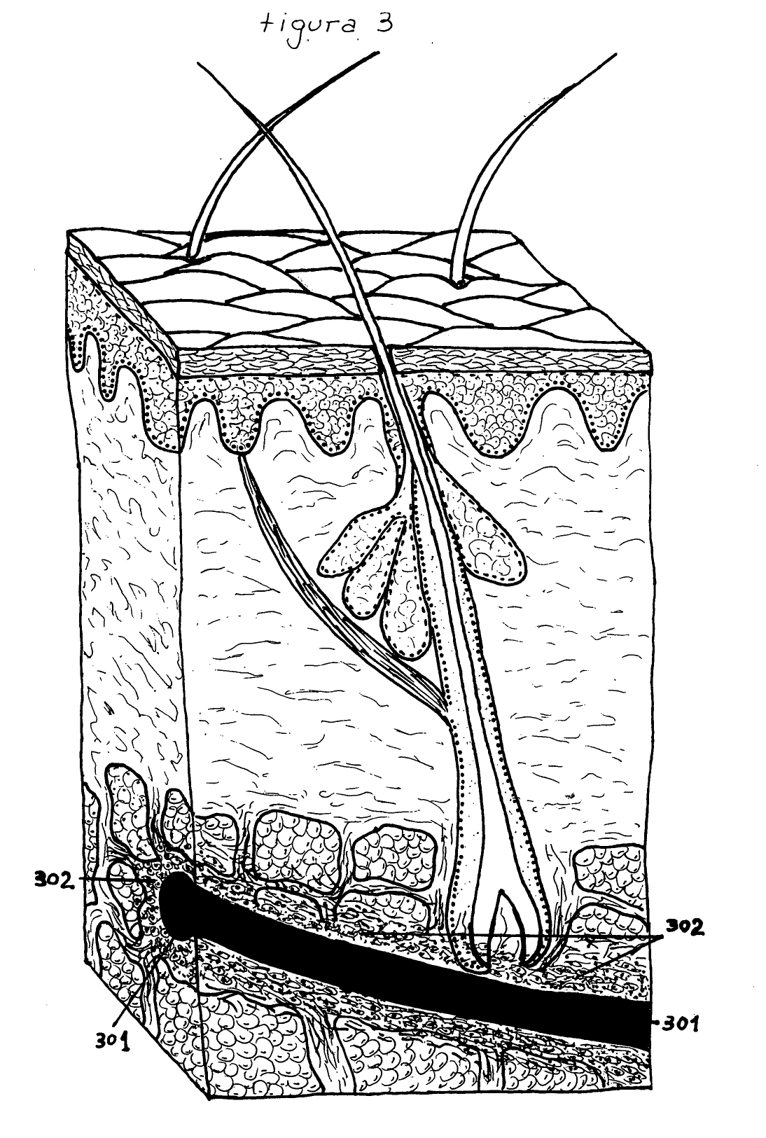

The organism forms a strange corpse type reaction (302) which isolates it, fixing it in the position shown in FIG. 3.

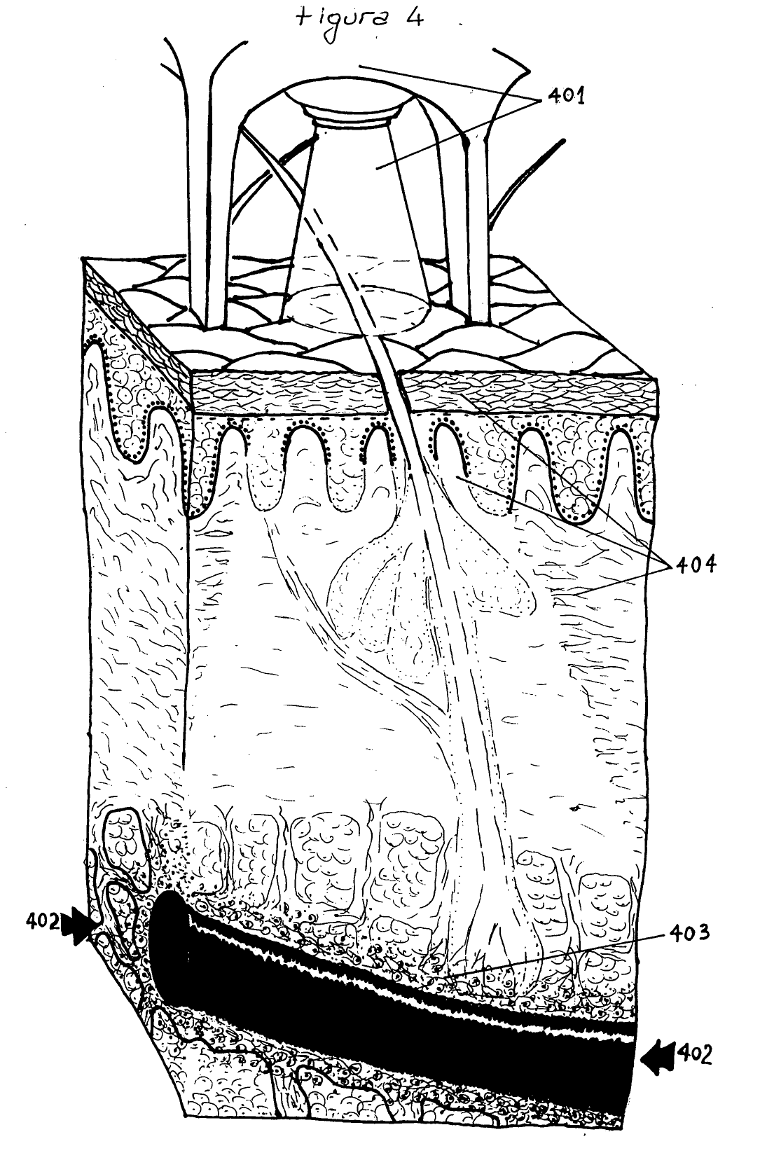

Once this fixing reaction has taken place, skin is stimulated externally with a monochromatic light beam (401) emitted by a L.A.S.E.R generator or other heat source which thermo-retracts it (402) in an innocuous way without affecting the integrity of the adjacent tissues as shown in FIG. 4.

This heat triggers the thread shortening reaction. Strange corpse reaction produced by the body totally surrounds the thread and it gets fixed by continuity to the skin deep layer or the mucous membrane lying over (403). Thread shortening is then mechanically transmitted to these tissues (404).

The thread form and its presentation allows it to be easily introduced under the skin (as when sewing fabric). It also allows a group assembling design for its placement, with the purpose of contributing to a better retraction with each one way or multiple direction strand, in order to handle the skin external retracted action (on a surface, or in one or various directions, according to convenience)

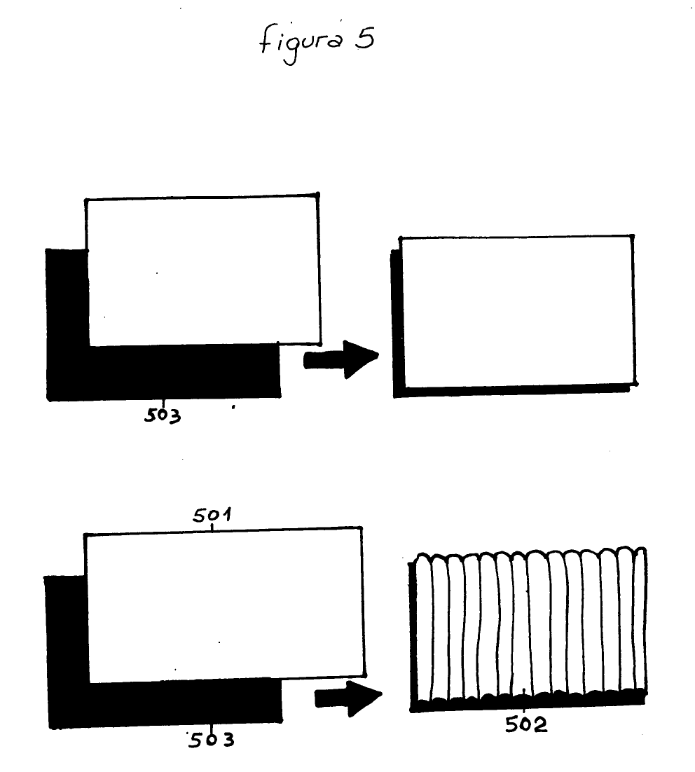

The thread joint action tends to counteract over the skin time-going-by stretching effect which when having stretched spreads over a larger surface (501) to cover the same original body area (503), thus the folding or wrinkling process taking place (502). See FIG. 5.

The thread can be gradually and progressively contracted, in successive heat stimulations. The material is externally invisible. It is inert, it neither produces waste nor varies its chemical composition with time. The strange corpse reaction caused by the body immunologic system isolates the thread as any other prosthetic element. The material only interacts mechanically with the human body. It is not dissolve or mixed with any human element.

1. Plastic mono-filamentary thermo-contractible surgical thread, featured as thermo-contractible and when introduced in the body it retracts to heat application and when interacting with adjacent tissues it grants them the same retracting property, dragging up with it the body parts involved.

2. The plastic mono-filamentary surgical thread of claim 1 featured as being texturized or of rough surface.

3. The plastic mono-filamentary surgical thread of claim 1 featured due to the fact that when gathering two or more threads they would form a cord or strand (multifilament).

4. The plastic mono-filamentary surgical thread of claim 1 featured as being texturized and when there is a gathering of two or more threads a cord or strand would be formed (texturized multifilament).
